(19) United States
(12) Patent Application Publication
(10) Pub. No.: US 2014/0076029 A1
(43) Pub. Date: Mar. 20, 2014

(54) LOAD-CONTROLLED ACCELERATED WEAR TESTING SYSTEM FOR HEART VALVE PROSTHESES AND OTHER CARDIOVASCULAR DEVICES

(71) Applicant: Shouyan Lee, Rancho Santa Margarita, CA (US)

(72) Inventor: Shouyan Lee, Rancho Santa Margarita, CA (US)

(21) Appl. No.: 13/941,334

(22) Filed: Jul. 12, 2013

Related U.S. Application Data
(60) Provisional application No. 61/671,541, filed on Jul. 13, 2012.

(51) Int. Cl. A61F 2/24

(52) U.S. Cl. CPC A61F 2/2472
USPC 73/37

(57) ABSTRACT

A load-controlled accelerated wear testing system to test cardiovascular prostheses that enables a user to tune pulse waveform by adjusting the output loading pressure of a linear motor driving the testing system. A portion of the testing system is open to the atmosphere during testing, allowing a relatively lower threshold pressure necessary to drive fluid across the prosthesis during testing. The contemplated tester can be set up in an array of multiple testers each capable of individual tuning and data-collection without undesirably cross-talking. Its novel feature of single-block construction out of a transparent material also allows direct visualization of the prosthesis during testing.

### CROSS-REFERENCE TO RELATED APPLICATIONS

This application claims priority to U.S. Provisional Pat. Application No. 61/671,541, filed on Jul. 13, 2012, now pending, which is hereby incorporated by reference in its entirety. This application also contains subject matter similar to that disclosed in Applicant's own Published Patent Application No. US2011/0303026 A1, now pending, entitled Portable Multifunction Cardiac Simulator and Heart Valve Tester, which is hereby expressly incorporated by reference as part of the present disclosure.

### BACKGROUND OF THE INVENTION

(1) Field of the Invention

The field of the present invention relates generally to prosthetic valve testers, and more particularly to an accelerated wear tester for accelerated wear testing (AWT) for prosthetic heart valves and other cardiovascular devices such as stents and percutaneous valves.

(2) Description of Related Art Including Information Disclosed Under 37 CFR 1.97 and 1.98

The advent of the prosthetic heart valve has provided many patients with both improved quality of life and increased longevity. The primary function of a prosthetic heart valve is to act as a check valve, opening to permit antegrade blood flow and closing to prevent retrograde flow, about one hundred thousand times per day. The valve elements move in response to a threshold pressure gradient in one direction, allowing flow through the valves, while closing in the opposite direction, preventing reverse flow below the threshold gradient pressure.

Prosthetic heart valves go through extensive testing and quality checks because failure of the valves in vivo can have catastrophic results. Certain characteristics such as durability, and proper fluid flow, are rigorously tested before a valve is deemed fit. Accelerated wear testing (AWT) is used to evaluate the durability of a prosthetic heart valve design. Current AWT testers on the market have several short comings: 1) they are difficult to tune in relation to valve opening and shape of the pressure waveform under ISO 5840 standard; 2) the peak pressure difference across a closed valve is quite high in order to comply with ISO 5840, 3) the devices are too large because of the requirements for a certain volume of fluid, size of motor, and the need for plumbing, 4) they are overly complex utilizing compliance modules to tune the pressure waveform and restrictions in the plumbing.

In addition, typical AWT testers are known for its efficiency in testing multiple valves at the same time in a single device. Each valve to be tested, however, has its own characteristics because of size, thickness, and other known and unknown factors (especially true for tissue valves, such as bovine tissue valves). Therefore, when tuning prior art AWT testers, all valves in the AWT tester is affected, and the problem of cross-talking results. It is particularly important to be able to tune the pressure waveform on each valve separately from other test valves.

There remains a continuing need for an AWT tester system that is relatively easier to tune, lightweight, portable, and allows concurrent testing on multiple prosthesis where the pulse waveform of each prosthesis can be individually and independently adjusted.

All referenced patents, applications and literatures are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply. Also, the invention may seek to satisfy one or more of the above-mentioned desires. Although the present invention may obviate one or more of the above-mentioned desires, it should be understood that some aspects of the invention might not necessarily obviate them.

### BRIEF SUMMARY OF THE INVENTION

An object of the present invention is to provide a load-controlled accelerated wear testing device that is individually tunable as to each prosthesis, with a frequency between 30-2400 cycles/min, producing constant, dynamic or random pressure waveforms in the system.

Another object of the present invention is to have an AWT tester having a linear motor where variables (e.g., current, pressure) of the linear motor's output force is adjustable (hence, load-controlled) during valve testing to achieve desired pressure waveforms under ISO 5840.

Another object of the present invention is to provide a load-controlled accelerated wear testing device wherein the linear actuator produces a driving pressure waveform within the fluid of the tester of between −50 to 150 mmHg.

Another object of the present invention is to provide a load-controlled accelerated wear testing device wherein the linear actuator produces a driving pressure waveform within the fluid of the tester of between −100 to 200 mmHg.

Another object of the present invention is to provide an AWT tester where an actuator with relatively small output would be sufficient to achieve the desire pressure waveform to fulfill ISO 5840 requirement, by having certain portion of the test fluid open to the atmosphere.

Another object of the present invention is to provide a load-controlled accelerated wear testing device wherein the cardiovascular and other prosthetic valves being tested can be directly observed during the test, and verified, for proper functioning.

Another object of the present invention is to provide a load-controlled accelerated wear testing device wherein the device itself is compact in size, is easily assembled or disassembled, and is easy to use.

Another object of the present invention is to provide a load-controlled accelerated wear testing device that tests one prosthetic valve per motor to prevent cross-talk between test subjects.

Another object of the present invention is to provide a load-controlled accelerated wear testing device that is sufficiently modular so as to be scalable such that the system can accommodate one valve, or a plurality of valves, conveniently and efficiently, while still being able to tune each valve separately from other valves.

Another object of the present invention is to provide a load-controlled accelerated wear testing device that is free from using compliance modules.

Another object of the present invention is to provide a load-controlled accelerated wear testing device where the resulting waveform is recorded and transmitted for analysis.

The present invention achieves its objects by providing a modular system of load-controlled accelerated wear testers where certain portion of each tester is exposed to the atmosphere. The manners in which the invention achieves its objects and other objects which are inherent in the invention will become more readily apparent when reference is made to the accompanying drawings wherein like number indicate corresponding parts throughout.

### BRIEF DESCRIPTION OF THE DRAWINGS

It should be noted that the drawing figures may be in simplified form and might not be to precise scale. In reference to the disclosure herein, for purposes of convenience and clarity only, directional terms, such as, top, bottom, left, right, up, down, over, above, below, beneath, rear, front, distal, and proximal are used with respect to the accompanying drawings. Such directional terms should not be construed to limit the scope of the invention in any manner.

FIG. 1 is a cross sectional view of a load-controlled accelerated wear tester illustrating the components of the tester wherein the test valve is mounted in a direction to allow a clock-wise flow of the fluid within, according to a preferred embodiment of the present invention.

FIG. 2 illustrates the four conduits present in FIG. 1, and the direction each conduit is bored. Although each specific direction is not critical, it is presented here to facilitate description of various components.

FIG. 3 is a cross sectional view of the load-controlled accelerated wear tester from FIG. 1, except wherein the test valve is mounted in a direction to allow a counter clock-wise flow of the fluid within, according to a preferred embodiment of the present invention.

FIG. 4A is a perspective view of the valve holder as shown in FIGS. 1, 3, 5, 7, 8, and 9. When it is inserted into the tester body, it would need to be rotated 45 degrees so its two side openings can align (top-to-bottom alignment) with conduit 82. This figure does not show the necessary hardware to fasten it to the tester body. One skilled in the art would have immediately recognized what type of hardware is needed, from looking at FIG. 1 and also from looking at the Applicant's related patent application cited herein.

FIG. 4B is a perspective view of another embodiment of the valve holder in scaffold form. Tester body is shown in simplified form. This figure does not show the necessary hardware to fasten it to the tester body. One skilled in the art would have immediately recognized what type of hardware is needed, from looking at FIG. 1 and also from looking at the Applicant's related patent application cited herein.

FIG. 5 is a perspective view of the load controlled accelerated wear tester from FIG. 1 showing additional instruments installed, according to a preferred embodiment of the present invention.

FIG. 6 is a perspective view of an array of four load controlled accelerated wear tester of FIG. 5, showing the modular nature of the system according to a preferred embodiment of the present invention.

FIG. 7 is a perspective view of another embodiment of load controlled accelerated wear tester, showing an atmosphere chamber with a different type of lid, and the restriction being installed at a different location, according to a preferred embodiment of the present invention.

FIG. 8 is a perspective view of another embodiment of load controlled accelerated wear tester, having an optional compliance installed.

FIG. 9 is a cross sectional view of yet another embodiment of load-controlled accelerated wear tester having interconnecting and detachable tubing to hold the prosthesis, according to a preferred embodiment of the present invention.

FIG. 10 is a block diagram illustrating a preferred method of using a load-controlled accelerated wear testing device.

FIG. 11 illustrates in diagram form the load controlled accelerated wear testing Control System according to a preferred embodiment of the present invention.

FIG. 12 is a block diagram of a load-controlled accelerated wear testing system illustrating the connections between the elements of the system, including the tester, amplifier, DAQ box, Control box, signal controller, PWM controller, and computer according to a preferred embodiment of the present invention.

FIG. 13 shows a screenshot of the contemplated user interface, the screenshot shows AWT analysis of valve number 1, which is in tester 1 of an array of four testers. Notice peak pressure of the four cycles shown on the screen is kept under 120 mmHg, and wherein more than 5% of cycles numbers 1, 2, 3, and 4 are above 95 mmHg, per ISO 5840 requirement.

FIG. 14 shows a screenshot of the contemplated user interface, this screenshot shows AWT analysis of valve number 2, which is in tester 2 of an array of four testers. Notice peak pressure of the six cycles on the screen is kept under 120 mmHg, and wherein more than 5% of cycles numbers 1, 2, 3, 4, 5 and 6 are above 95 mmHg, per ISO 5840 requirement.

### DETAILED DESCRIPTION OF THE INVENTION

Referring now to the drawings wherein the showings are for purposes of illustrating a preferred embodiment of the present invention and not for purposes of limiting the same.

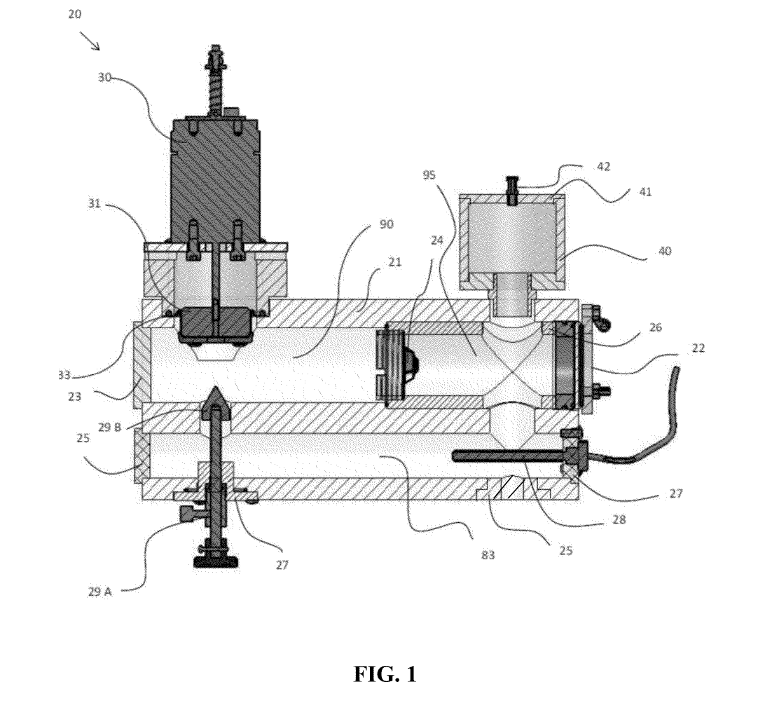

A first embodiment of the invention is load-controlled accelerated wear testing system composed of a tester 20, as depicted in FIG. 1, a single block of transparent material makes up the tester body 21, being compact in size, holds six or more liters of fluid, is disposed with viewing windows 22, 23 on both the distal and proximal ends (respectively) of the tester body 21 to enable direct observation of a test valve 24 (which may also be a tissue valve, a mechanical valve, a stent, a percutaneous valve). The tester body 21 contains a circuitous channel to hold a fluid to be driven by the linear actuator 30. Contemplated fluid includes incompressible testing fluid such as water, saline solution, organic solvent or other aqueous liquids.

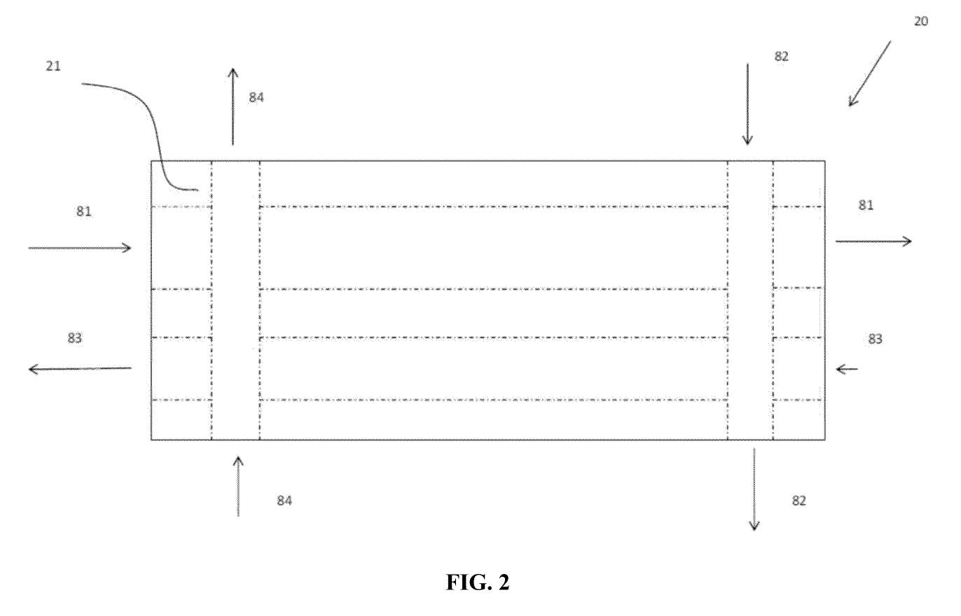

In FIG. 1, the circuitous channel is basically comprised of four intersecting straight bore-through conduits 81, 82, 83, 84 (please also refer to FIG. 2). The particular configuration as shown in FIG. 1 is made by boring through a single block of transparent acrylic (or plexiglass, or other transparent or non-transparent material) at appropriate places such that the four intersecting conduits are fluidly connected. As shown in FIGS. 1 and 2, the entry holes and exiting holes of conduits 81, 82, 83, and 84 are appropriately closed off to create the intended circuitous channel.

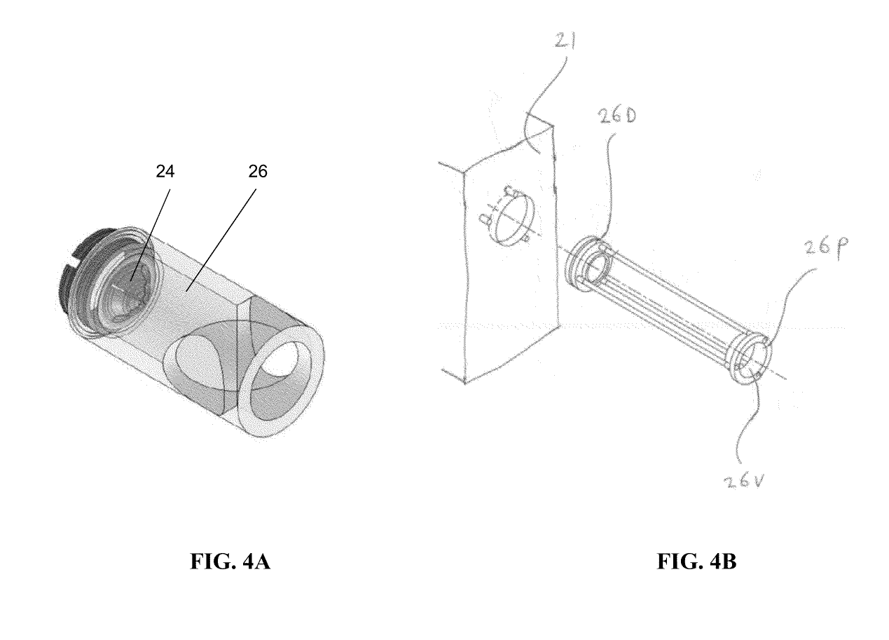

Conduit 81 is bored through from left to right. The entry hole on the left side is sealed with viewing window 23, which can be fastened to the tester body 21 by detachable means such as screws, screw blades, or simply plugs into the entry hole. The exiting hole of conduit 81 on the right side is sealed by viewing window 22 which is part of the prosthetic holder 24, and will be describe in further detail below and in FIGS. 4A and 4B.

Conduit 81 is fluidly connected to conduit 82, which can be created by boring through the tester body 21 in a top to bottom direction. The entry hole of conduit 82 is not completely sealed, and it connects to an atmospheric chamber 40. Conduit 81 is fluidly connected to the atmospheric chamber 40, such that fluid in conduit 81 can flow into the atmospheric chamber 40, which will be described in more detail below. The exiting hole of conduit 82 is sealed with a transparent window plug 25. Window plug 25 is completely solid and seals the exiting hole of conduit 82.

Conduit 82 is fluidly connected to conduit 83, which can be created by boring through the tester body 21 in a right to left direction. The entry hole of conduit 83 can be sealed with a simple plug such as previously described transparent window plug 25. Here, a plug 25 is not used, but instead, an optional temperature sensor 28 having a back plate 27 is used. The exiting hole of conduit 83 is shown as being sealed with another transparent window plug 25.

Conduit 83 is fluidly connected to conduit 84, such that fluid in conduit 83 can flow into conduit 84. Conduit 84 is in turn fluidly connected back to conduit 81, such that fluid in conduit 84 can flow into conduit 81, completing a circuitous flow. Conduit 84 can be created by boring through the tester body 21 in a bottom to top direction. The entry hole of conduit 84 can be sealed with a simple plug such as transparent window plug 25. Here, the embodiment shown in FIG. 1 has the entry hole sealed with a restriction 29A having stopper 29B and a back plate 27. Here, the restriction 29A is in the form of a manually adjustable stopper 29B to restrict the amount of flow passing between conduits 81 and 83. A user can turn the bottom part of the restriction in order to move the stopper 29B up and down, causing varied degrees of occlusion in conduit 84. In FIG. 1, the flow is completely restricted by the stopper 29B. In a preferred embodiment, the stopper 29B is left in a fully open or partially restricted position throughout the test, after proper adjustment and tuning of pressure waveform.

The exiting hole of conduit 84 is shown as being sealed with a linear actuator 30. Linear actuator 30 (though other forms of actuation could be used) has a piston 31 to cycle up and down. The piston is sealed around its periphery with a rolling diaphragm 33. Essentially, fluid is prevented from leaking out of the exiting hole of conduit 84 because of the rolling diaphragm 33 and the piston 31.

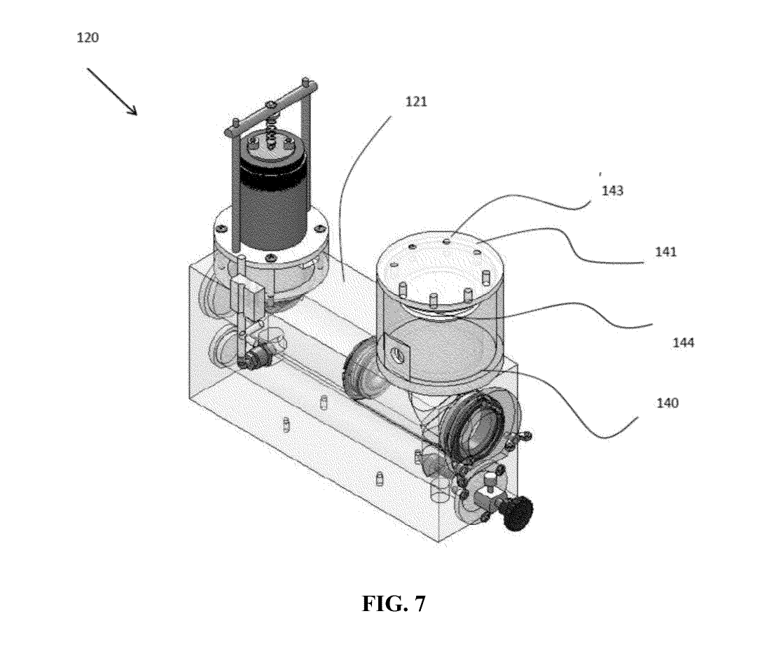

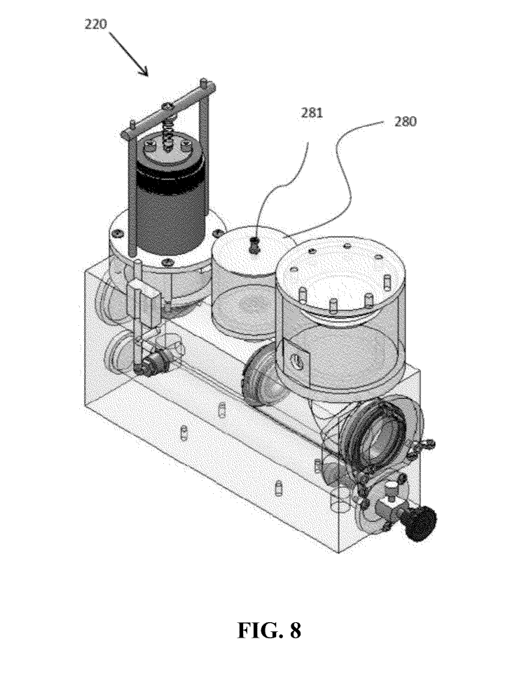

As described above, conduits 81, 82, 83, 84 make up the circuitous channel. The solid transparent block embodiment as described above uses a novel method of creating the desired circuitous flow by boring four intersecting conduits 81, 82, 83, and 84. This is the preferred method of manufacturing because of its relatively easy steps and low cost and it has less possibility for fluid leakage. The entry holes and exiting holes as described are advantageous as they provide a way to place instruments (e.g., restriction, pressure sensor, temperature sensor, heating rod) into the circuitous channel. Or, instead of having an instrument, the entry holes and exiting holes can act as viewing windows. This means that the contemplated instruments (e.g., restriction, pressure sensor, temperature sensor, heating rod) can be interchangeably placed at various places without limitation as to where it must be installed. For example, the embodiment of FIG. 1 has the restriction 29A installed at the entry hole of conduit 84. In the embodiment shown in FIGS. 7 and 8, the restriction is located where temperature sensor 28 is in FIG. 1.

Other known methods of manufacturing the tester body 21 are also contemplated, so long as the tester 21 has a circuitous channel, and there's a way to place a test valve inside of the circuitous channel, allowing the fluid in the circuitous channel to be driven by an actuator, and where a portion of the circuitous channel is open to the atmosphere.

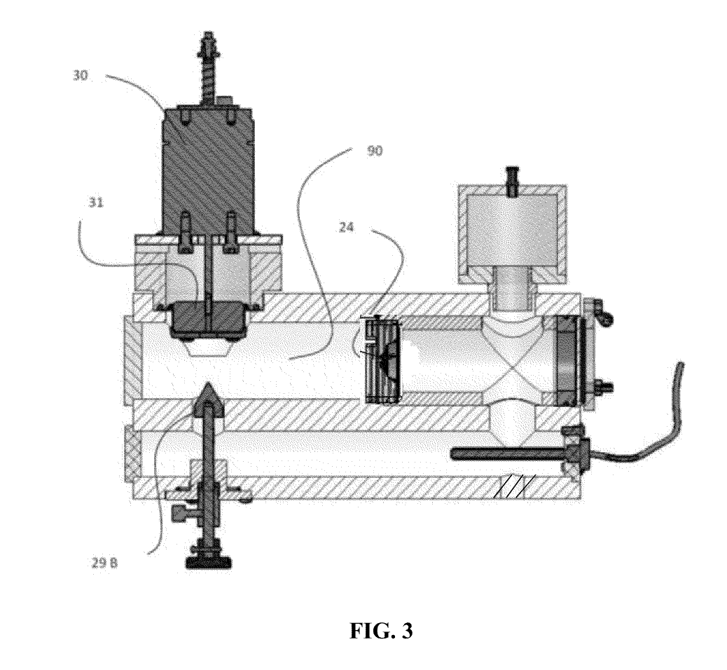

Referring now to FIGS. 4A and 4B, a detachable holder 26 can be removed from conduit 81 for mounting of the valve 24. Valve 24 is mounted within the detachable holder 26 so as to position the prosthetic valve 24 somewhere within conduit 81. The holder 26 as shown in FIGS. 1 and 3 are separately shown in FIG. 4A. Other embodiments of the holder 26 includes using a scaffold such as the one disclosed in the Applicant's Published Patent Application No. US2011/0303026 A1, now pending, which is hereby expressly incorporated by reference as part of the present disclosure. In that embodiment, the detachable holder 26 is a scaffold that holds the valve 24 at the scaffold's distal end 26D, while its proximal end 26P fastens onto the tester body 21. The scaffold has a transparent viewing window 26V.

Returning now back to the valve holder 26 as shown in FIGS. 1, 3, and 4A; as the viewing window 22 fastens onto the tester body 21, the viewing window 22 seals off the channel so no fluid can leak out around the viewing window 22. And during an un-mounting procedure, before removing the detachable holder 26 from the tester body 21, the tester 20 is first drained so fluid would not undesirably leak out from around the viewing window 22. The fluid is refilled into the circuitous channel only after the valve holder 26 and the viewing window 22 is returned and sealingly fastened to the tester body 21.

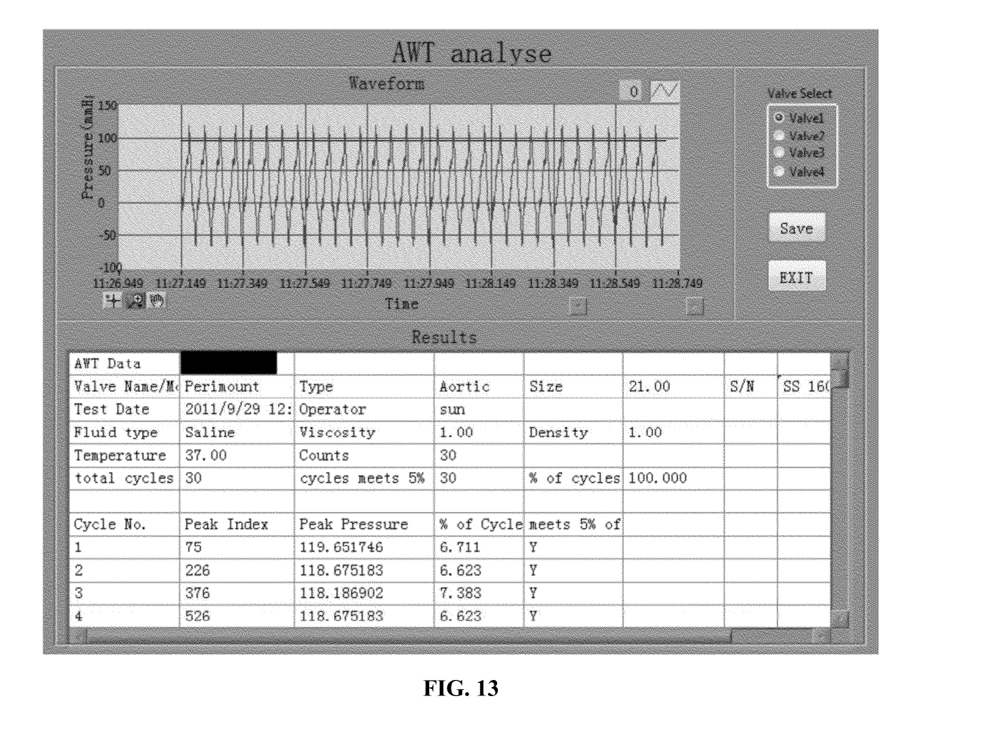

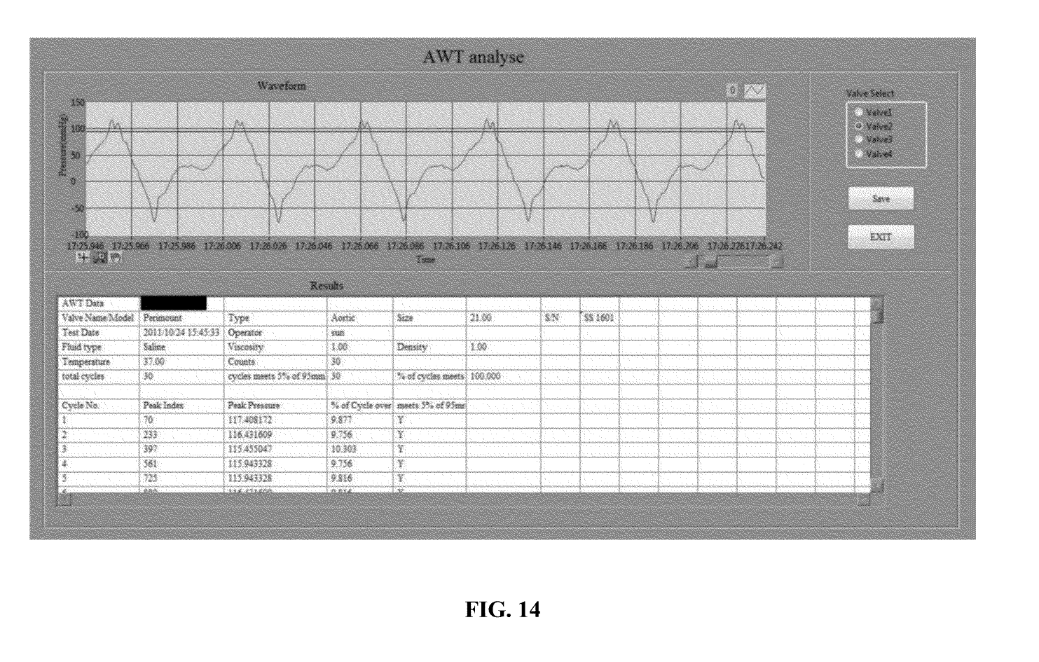

Referring now to the linear actuator 30 in the tester 20; linear actuator 30 is a motor operable at 30-2400 cycles/min, sufficient to produce a transvalvular pressure of between 120 mmHg and −50 mmHg pressure in each cycle. This pressure range meets the requirements for an aortic valve testing specified in ISO 5840. This range also meets the minimum ISO 5840 requirement of having at least 5% of each cycle being over 95 mmHg in transvalvular pressure. FIG. 13 shows an analysis waveform and data showing as such.

In a preferred embodiment, the tester 20 can have at least 5% of each cycle being over 95 mmHg in transvalvular pressure, whereby a user has tuned each cycle to a range within −100 to +200 mmHg; more preferably, within −75 to +150 mmHg; even more preferably, within −75 to +125 mmHg, and also preferably, within −50 to 150 mmHg, and most preferably, within −50 to 120 mmHg.

Furthermore, the contemplated inventive subject matter can use a very small peak pressure (i.e., 100-120 mm Hg, see FIG. 13) to pass fluid across the prosthetic valve 24. FIG. 13 shows pressure waveform of transvalvular pressure in six cycles, wherein each cycle has at least 5% being over 95 mmHg in transvalvular pressure, and each cycle peaked at no higher than 118 mmHg. In other contemplated embodiments, this can be achieved with each cycle peaked at no higher than 300 mmHg; more preferably, with each cycle peaked at no higher than 200 mmHg; still more preferably, with each cycle peaked at no higher than 150 mmHg; and also preferably, the driving load selectively applied is between 50 to 300 mmHg above the atmospheric pressure, still more preferably, the driving load selectively applied is between 100 to 200 mmHg above the atmospheric pressure, and further preferably, driving load results in a pulse waveform that fluctuates between about 120 mmHg and minus 50 mmHg, and most preferably, with each cycle peaked at no higher than 130 mmHg. Conversely, in prior art AWT testers, because much pressure is needed to charge compliance modules and to overcome restrictions, the peak pressure is considerably higher. In one contemplated method, a user can easily tune into the desired pressure range by directly tuning the pressure using a pulse wave modulation controller.

During each cycle of piston 31 movement, the linear actuator 30 drives a pressure waveform in the circuitous channel and across the test valve 24. As the actuator 30 loads pressure into the circuitous channel, pressure regions are created. With respect to the embodiment of FIG. 1, the circuitous channel can be described to generally comprise of two pressure regions: the load region 90 and the static pressure region 95. The loading region 90 is generally upstream of the prosthetic valve opening. In FIG. 1, the direction of flow is clock-wise (determined by the direction of valve 24), and load region 90 is located left of the valve 24. Static pressure region 95, on the other hand, is located to the right of the valve 24.

During a pre-loading period, that is, before the piston moves downward to apply a loading pressure (i.e., a driving load), the static pressure region 95 is contemplated to remain at substantially atmospheric pressure because it is fluidly connected to the atmosphere. As the piston moves downward to apply a loading pressure (i.e., loading period), pressure in the load section 90 builds. Before the pressure builds to reach above a threshold value to open the testing valve 24, the static pressure region 95 is contemplated to remain at substantially atmospheric pressure (the meaning of which includes atmospheric pressure plus the height of the fluid, if any, in the atmospheric chamber 40). When the threshold value is reached, the testing valve 24 opens, fluid passes through the testing valve 24 from left to right, pressure in the load section 90 is reduced, and pressure in the static pressure region 95 suddenly surges, causing the prosthesis 24 to close. As fluid travels down into conduit 83, pressure in static pressure section drops and returns to substantially atmospheric pressure.

As one of ordinary skill in the art would recognize, the threshold pressure that sufficiently opens the prosthesis 24 is a pressure level sufficiently larger than static pressure in the static pressure region 95.

In the preferred embodiments, a user can tune the waveform by directly adjusting variables (e.g., pressure, current, speed) that affect the output force of the linear motor 30. In effect, the linear motor 30 directly applies loading pressure on the prosthesis 24.

A key component of the inventive subject matter is for the tester 20 to have a portion being the static pressure region 95. Known heart valve testers and AWT testers, however, are closed systems that do not allow any portion of its fluid flow to open to the atmosphere. Having such static pressure region 95 is contrary to common industry practice and understanding. For decades, heart prosthesis has been tested under a closed system having compliance modules and resistance modules, with the entire system under substantially much larger pressure. It is commonly known in the industry to supply more and more compliance modules, resistance modules, and to test the prosthesis under systemic pressure that is more and more difficult to tune. The inventor, however, have surprisingly discovered the advantages of a testing method that is a complete reverse of what the prior art teaches and opposite of industry development trends. This novel method allows easy tuning of desirable waveforms by a user.

A critical component in the contemplated tester 20, therefore, is the atmospheric chamber 40. The atmospheric chamber 40 has an optional lid 41, and the lid has a fitting 42. The embodiment as shown in FIG. 1 has a lid 41 with a fitting 42, whereas the embodiment as shown in FIGS. 7 and 8 have a lid 141 that is raised on multiple pegs 143, allowing a gap 144 to open to the atmosphere.

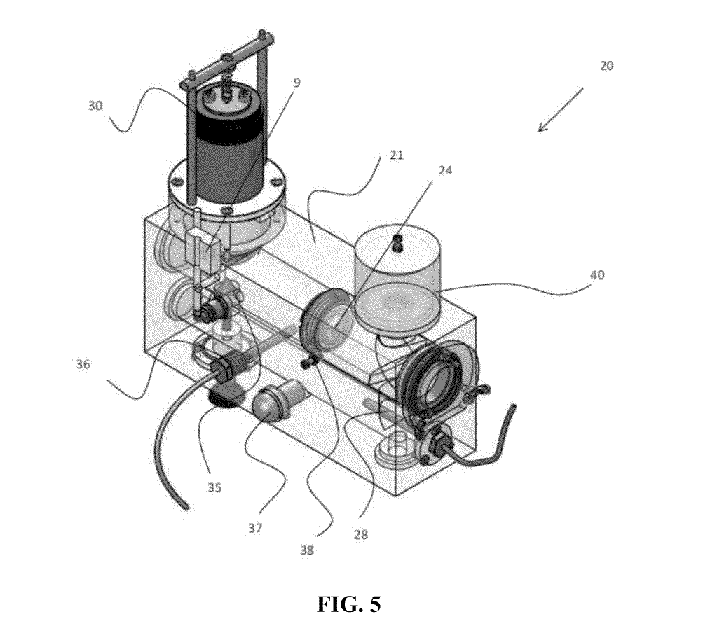

In the embodiment of FIG. 1, the instruction of operation is as s follows: 1) After the testing valve 24 is placed into the circuitous channel by the valve holder 26, begin filling fluid through a fill port 38 (see FIG. 5), or by pouring fluid into the atmospheric chamber 40 with the lid off. 2) Stop filling of fluid when fluid level reached the middle to lower part of the atmospheric chamber 40. 3) Place lid 41 on top of atmospheric chamber 40, and twist close the fitting 42. 4) Now a user may tilt the tester 20 around to remove any air bubbles in the system by moving the air bubble to the atmospheric chamber 40. This can be done without spilling fluid from the atmospheric chamber 40 because the fitting was twisted shut. 5) Once all air bubbles are removed from the circuitous channel, twist open the fitting 42 so the interior of the atmospheric chamber 40 will now remain in fluid communication with the atmosphere during the test. Alternatively, remove the lid 41 from the atmospheric chamber 40 altogether during the test.

Preferred embodiments would have some type of lid to prevent fluid spills during testing.

The static pressure section 95 is maintained at substantially constant pressure by means of this atmospheric chamber 40 that is contemplated to be in fluid communication to the atmosphere. As discussed, the atmospheric chamber 40 does not require a complete lid-less design, so long as it is somehow open to the atmosphere. Although the static pressure region is described as having a substantially constant pressure, it should be understood that the pressure in the static pressure section 95 does surge when a pressure load pushes fluid pass through the valve 24 as shown in FIG. 1. The key is that the static pressure section 95 is in fluid communication with the atmosphere, allowing relatively substantial constant static pressure in the static pressure section 95.

Again, it should be understood that the contemplated tester 20 does not require a compliance module, as typical AWT testers do. Such known compliance module, however, can be optionally provided to the contemplated system 20. For example, a compliance chamber 280 can be disposed on top of load section 190 in FIG. 8, and be in fluid communication with load section 90. As is known in the art, the fitting 281 on top of the compliance 280 will remain shut during testing. This optional compliance chamber 280 can minimize noise. In a preferred embodiment, this optional compliance chamber 280 is not for waveform adjustment.

FIG. 3 shows the same tester 20 as in FIG. 1, except that the valve 24 is installed facing a direction opposite of that in FIG. 1. In this configuration, the flow of fluid during operation would be counter-clockwise. As in FIG. 1, here the flow is completely restricted by the stopper 29B. This is merely to show that the stopper 29B is capable of upward movement by manual adjustment. During operation, however, the stopper 29B must be left in a fully open or partially restricted position throughout the test, after proper adjustment and tuning of pressure waveform.

Imagine the stopper 29B being positioned lowered than as shown in FIG. 3, allowing a fully open or partially restricted flow pass the stopper 29B. As the piston 31 moves downwardly to apply a loading pressure to loading region 90, the fluid is pushed pass restriction plug 29B into conduit 83. As the piston 31 moves up, a negative pressure is created in the loading section 90, and the valve 24 opens as a result.

Referring now to FIG. 5, as described above, the contemplated load-controlled accelerated wear tester 20 can be equipped with additional instruments necessary for data collection and other functions. Here, the tester 21 can include a pressure transducer 9 to detect and collect pressure data. Also shown is a heating element 36 to heat the fluid. Tester 20 also has a fill port 38 and a drain 37 in the body of the tester body 21. The drain 37 and fill port 38 can be connected to a reservoir (not shown) so that an optimal fluid level can be kept in the tester system 20 during testing.

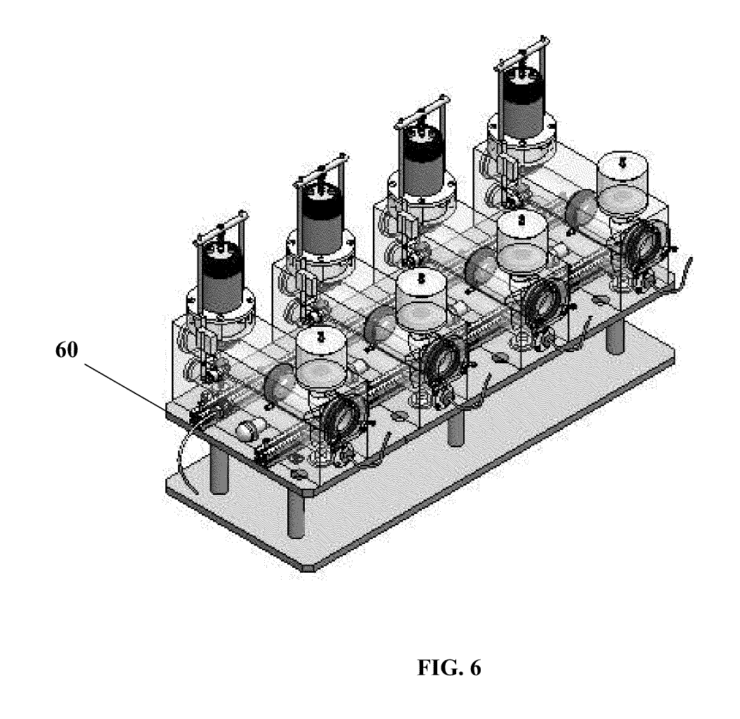

Further contemplated advantages of the invention include the ability to scale up AWT testing without the problem of cross-talking between multiple testing valves. As illustrated in FIG. 6, four of the tester 20 from FIG. 1 are arranged on a platform 60 and connected to the same electronics (not shown). Although FIG. 6 shows only four such testers 20, one skilled in the art would immediately recognize the scalability of this arrangement. Multiple testers 20 can be set up in such array, and each tester 20 can be individually and separately tuned to achieve desirable pressure waveform. Essentially, the preferred embodiment is a one-motor, one-valve system where a single motor is responsible for only one testing valve. This eliminates the prior art problem of cross-talking between multiple valves driven by the same motor. All testers 20 in a group may share the same fluid reservoir (not shown).

A further contemplated tester 120 is shown in FIG. 7 where conduit 82 and 84 are not entirely bored through the tester body 121. Also, restriction is installed at the entry hole of conduit 83. The atmospheric chamber 140 in FIGS. 7 and 8 is shown with a side port (not numbered). This optional side port can be connected to a main reservoir for filling and maintenance fluid level in tester 120.

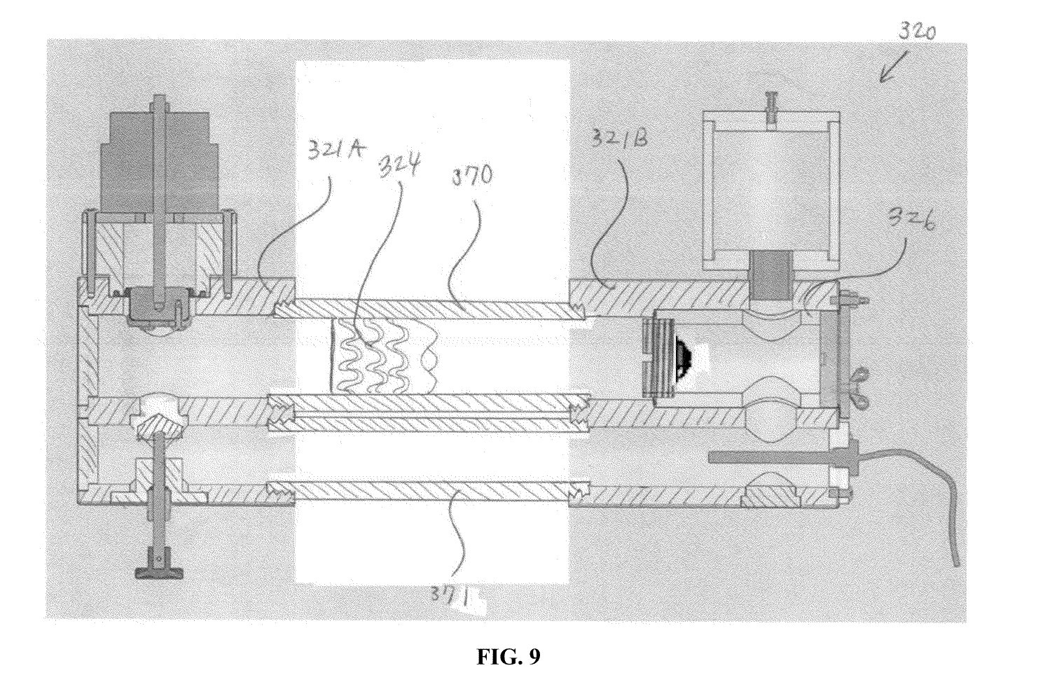

Thus far, the specification has described a single-block configuration made of acrylic, plexiglass, or other transparent or nontransparent material. Although a single-block design is the preferred embodiment, another embodiment contemplates having the tester 20 physically separable into two pieces, where the two pieces are connected by tubing. Referring now to FIG. 9, tester 320 has tester bodies 321A, 321B connected via detachable tubing 370, 371. The key component of this design is for tubing 370 to hold a stent or a subcutaneous valve 324 for testing. As shown, the rest of the tester 320 remains similar to earlier embodiments. Tubing 370, 371 is preferably made of transparent material to assist viewing of the valve 324 during testing, and can be fastened onto tester bodies 321A, 321B via screw blades or other known fastening means. An optional valve holder 326 can be used to hold a valve as described previously. Alternatively, tubing 370 can act as a valve holder to hold a valve. In yet another embodiment, the valve holder 326 is unnecessary and can be removed from the tester body 321B during testing.

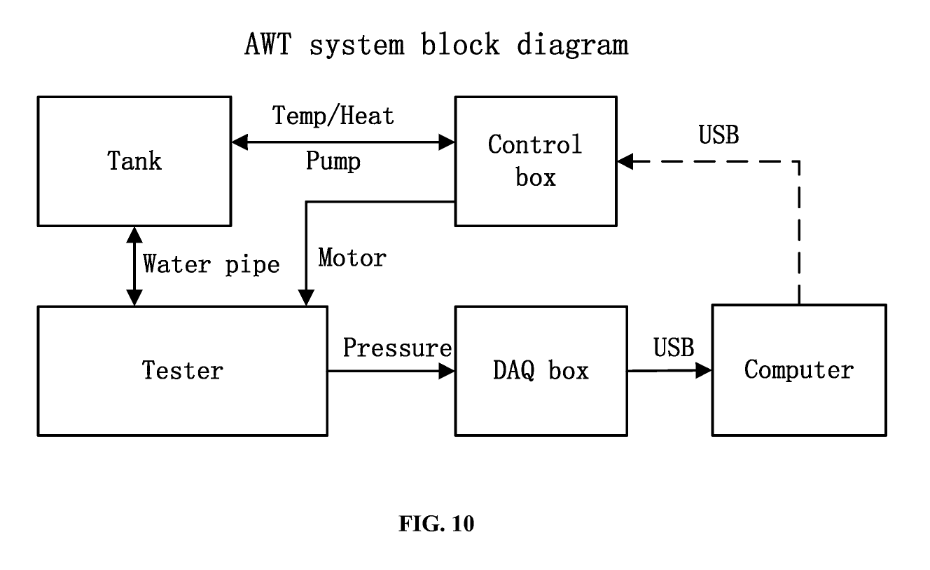

Yet further embodiments of the invention are a load-controlled accelerated wear testing system having the tester with associated electronics as illustrated in FIG. 10. The block diagram depicts connections between elements of the system. The primary system element is the control box. The control box is powered by a 150 W transformer that may run on either 220V or 110V systems. The Control box is capable of powering at least four accelerated wear testers simultaneously, including the linear motor, heater, thermometer, and pressure transducer of each system. Each tester is connected to single DAQ (data acquisition) box, which outputs data via USB to a Computer containing a DAQ card. The Computer is also connected to the Control box via USB cable.

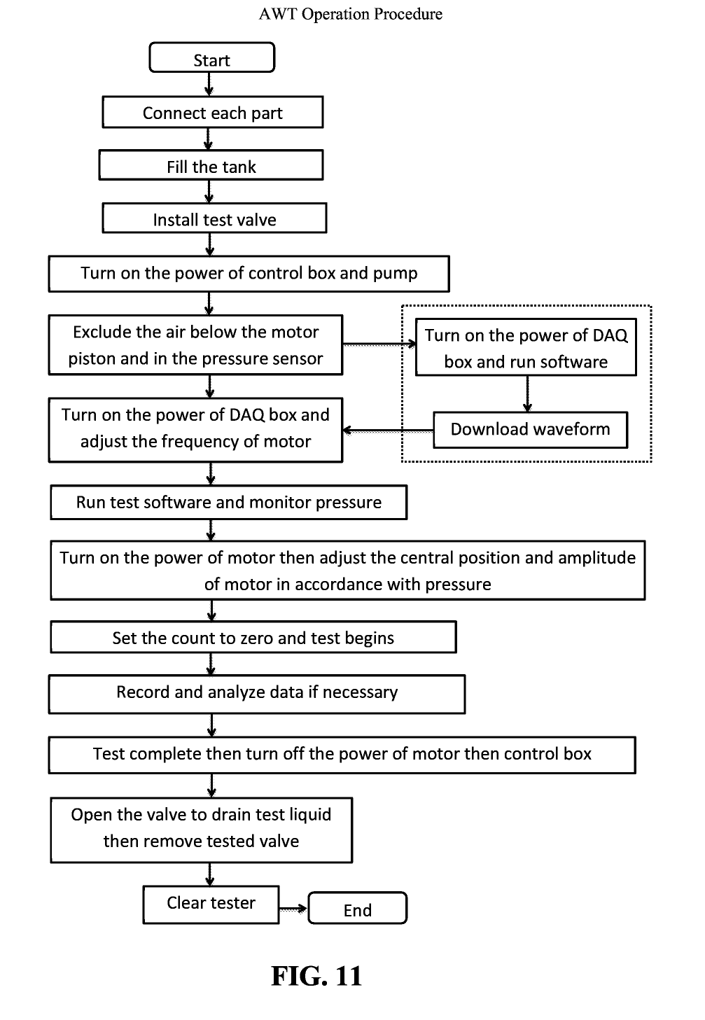

FIG. 11 illustrates steps in a preferred method of testing a valve. It is operated by first assembling the system, filling the tank with 6 L of solution, installing the test valve, turning on power to control box and pump, eliminating air below the motor piston and the pressure sensor, turn on the power to the DAQ box, adjust the frequency, monitor pressure, calibrate offset and sensitivity of pressure transducers, turn on power of motor, adjust center and amplitude of motor in accordance with pressure, set the cycle count to zero and begin recording test data, analyze test data, power off the system, drain test liquid back to tank, remove test valve, clear tester.

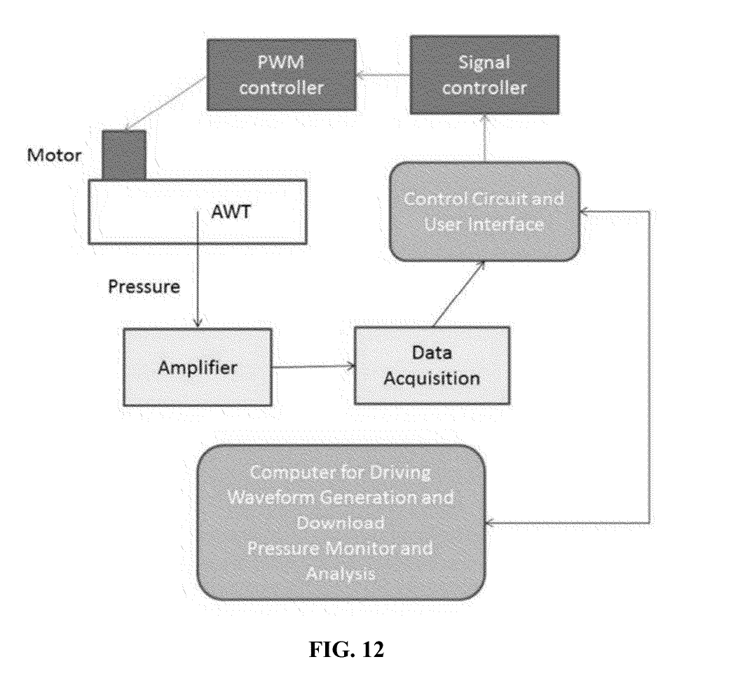

Another embodiment of the load-controlled accelerated wear testing system includes usage of software. Referring now to FIG. 12, the software possesses 7 tab options including: Monitor, Data Record, DO, Serial, Calibration, Valve inf. and Analyze. Monitor is used for real-time monitoring of the pressure signal; Data Record is used to record the waveform data for analysis; DO is a reserved function for automatic control, Serial is to download waveform data to the control circuit, Calibration is used to calibrate the pressure transducer and adjust the offset; Valve inf. contains valve and test information; Analyze is data processing per ISO 5840. Here, the linear actuator is electrically connected to a pulse wave modulator controller. The pulse wave modulator controller specifies the amplitude and frequency of the linear actuator motion. The pressure transducer measures the pressure immediately near the linear actuator. The pressure transducer is electrically coupled to an amplifier and then to a data acquisition system. The data is then downloaded to a computer with a user-interface enabling the operator to selectively adjust the driving load on the prosthesis. In one embodiment, the user selectively adjusts the driving load to between 5-500 mmHg over atmospheric pressure; in a preferred embodiment, between 50-300 mmHg; in yet other preferred embodiments, 100-200 mmHg; still more preferably, 100-150 mmHg; most preferably, 100-120 mmHg.

The data is analyzed, output generated, and transmitted back to the control circuit which transmits via signal controller to the pulse wave modulator controller. Because the linear actuator 30 of the load section 90 is powered by direct current, the driving waveform is directly related to the pressure waveform thereby simplifying tuning of the pressure waveform, to constant, random or other pressure waveform species. The system is further disposed with a pause button for convenience. The recorded waveform data enables the system to provide a quantitative assessment of the tested prosthetic valve.

As discussed above, a key component of the inventive subject matter is the ability to electronically tune a pressure waveform. For decades, it has been common practice to tune a AWT testing system to achieve the correct waveform by mechanical means, that is, by adjusting compliance modules and resistance modules in a testing system. Such mechanical means to tune the waveform are rather difficult and excessively cumbersome. In one commonly known practice, it requires a user to increase volume (volume-control) or displacement (displacement-control) to charge the compliance modules and overcome resistance to achieve desired waveform. In such practice, a rather large motor is needed in order to charge the compliance modules and to force fluid across the prosthesis 24. Prior art devices are thought to better simulate a physiological system (e.g., narrowing vessels, occlusions in the body after the blood leaves the heart, organs). Surprisingly, the inventor has discovered a cost-effective AWT method that requires no compliance modules while meeting ISO 5840 requirements.

It will be understood that various modifications can be made to the various embodiments of the present invention herein disclosed without departing from the spirit and scope thereof. For example, various devices are contemplated as well as various types of construction materials. Also, various modifications may be made in the configuration of the parts and their interaction. Therefore, the above description should not be construed as limiting the invention, but merely as an exemplification of preferred embodiments thereof. Those of skill in the art will envision other modifications within the scope and sprit of the present invention as defined by the claims appended hereto.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims therefore include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Thus, specific embodiments and applications of load-controlled accelerated wear testing system have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Insubstantial changes from the claimed subject matter as viewed by a person with ordinary skill in the art, now known or later devised, are expressly contemplated as being equivalent within the scope of the claims. Therefore, obvious substitutions now or later known to one with ordinary skill in the art are defined to be within the scope of the defined elements. The claims are thus to be understood to include what is specifically illustrated and described above, what is conceptually equivalent, what can be obviously substituted and also what essentially incorporates the essential idea of the invention. In addition, where the specification and claims refer to at least one of something selected from the group consisting of A, B, C . . . and N, the text should be interpreted as requiring only one element from the group, not A plus N, or B plus N, etc.

What is claimed is:

1. A load-controlled accelerated wear testing system to test a prosthesis, comprising:
a tester body having a circuitous flow channel, said channel providing a fluidly circuitous path within said channel;
an actuator coupled to said channel to exert a driving load upon a fluid and cause a fluid flow within said channel;
a detachable prosthesis holder disposed within said circuitous path to hold said prosthesis within said circuitous path;
wherein the channel is coupled to an opening opened to the atmosphere during the accelerated wear testing; and
wherein the actuator is at least one of A) user-adjustable by selectively adjusting the driving load, B) close loop force-controlled, and C) user-adjustable by tuning a pressure using a pulse wave modulator coupled to the actuator.

2. The load-controlled accelerated wear testing system as recited in claim 1, further comprising:
a load region disposed within the channel disposed between the prosthesis holder and the actuator;
a static pressure region disposed within the channel between the prosthesis holder and the opening;
wherein the actuator has a piston to load a pressure of the fluid in the load region which in turn drives the fluid flow within the circuitous channel; and
wherein the static pressure section is at substantially atmospheric pressure before a liquid in the load section flows from the load section to the static pressure section.

3. The load-controlled accelerated wear testing system as recited in claim 2, further comprising an atmospheric chamber branched out from said channel and is fluidly connected to the channel, and wherein the opening is disposed on top of the atmospheric chamber, and wherein the substantially atmospheric pressure of the static pressure section is an atmospheric pressure plus a pressure of any amount of fluid in the atmospheric chamber, before there is movement of fluid from the load section to the static pressure section.

4. The load-controlled accelerated wear testing system as recited in claim 3, wherein the cardiovascular prosthesis is one selected from the group consisting of a tissue valve, a mechanical valve, a stent, and a percutaneous valve.

5. The load-controlled accelerated wear testing system as recited in claim 4, further comprising an electrical control and measurement component, the electrical control and measurement component is comprised of:
a pulse wave modulation controller electrically coupled to the actuator;
a pressure transducer disposed in the circuitous channel and electrically coupled to an amplifier;
a temperature sensor disposed in the circuitous channel to detect a temperature of the fluid;
a heater disposed in the circuitous channel to heat the fluid;
a computer electrically coupled to a data acquisition system and to the pulse wave modulation controller, said computer having a user-interface allowing an operator to selectively adjust the driving load directly placed on the prosthesis.

6. The load-controlled accelerated wear testing system as recited in claim 5, wherein the actuator is a linear actuator, and further comprising a rolling diaphragm sealingly disposed around the piston.

7. The load-controlled accelerated wear testing system as recited in claim 6, wherein the tester body is a solid block of transparent material, allowing direct visualization of the circuitous channel, and further comprising a proximal viewing window disposed at a proximal end of the tester body, and a distal viewing window disposed at a distal end of the tester body; wherein both the proximal window and the distal window allow a viewing path substantially parallel to a travel path of the fluid as it passes through the prosthesis.

8. The load-controlled accelerated wear testing system as recited in claim 6, wherein more than one of said tester body is coupled to the electrical component in an array, each tester body capable of individually and separately tuning the driving load in each actuator of each tester body.

9. The load-controlled accelerated wear testing system as recited in claim 6, wherein the tester body is comprised of two pieces such that separating the two pieces effectively allows the load section to be detachable from the static pressure section; and further comprising two detachable tubing to detachably and fluidly connect the two pieces, wherein one of the two tubing is the detachable prosthetic holder.

10. The load-controlled accelerated wear testing system as recited in claim 7, wherein no compliance module is coupled to the circuitous channel.

11. The load-controlled accelerated wear testing system as recited in claim 11 further comprising a restriction in the circuitous channel

12. The load-controlled accelerated wear testing system as recited in claim 5, wherein the electrical component allows a user to fine-tune a pulse waveform by adjusting the driving load of the actuator, and the pulse waveform being approximately between −100 to +200 mmHg.

13. The load-controlled accelerated wear testing system as recited in claim 13, wherein the pulse waveform being approximately between −50 to 150 mmHg.

14. The load-controlled accelerated wear testing system as recited in claim 9, wherein more than 5% of a cycle in the waveform is above 95 mmHg, and the waveform has a peak pressure not exceeding 200 mmHg.

15. The load-controlled accelerated wear testing system as recited in claim 14, wherein the peak pressure does not exceed 130 mmHg.

16. A method of testing accelerated wear of a prosthesis, the method comprising:
providing a circuitous channel having a fluid within, the circuitous channel having a load region, a prosthesis holder holding a prosthesis, and a static pressure region;
providing an opening opened to the atmosphere coupled to the static pressure region thereby causing the pressure in the static pressure region during a pre-loading period to remain substantially at atmospheric pressure;
providing a close loop force or force equivalent variables-controlled linear actuator having a piston, wherein the linear actuator is coupled to the load section, to apply a driving load to the load section during a loading period;
selectively apply the driving load at between 5 to 500 mmHg above the atmospheric pressure to drive the fluid across the prosthesis, and achieving at least 5% of each cycle at under 95 mmHg.

17. The method as recited in claim 17, wherein the driving load selectively applied is between 50 to 300 mmHg above the atmospheric pressure.

18. The method as recited in claim 17, wherein the driving load selectively applied is between 100 to 200 mmHg above the atmospheric pressure.

19. The method as recited in claim 17, further comprising the step of selectively adjust the driving load so a resulting pulse waveform fluctuates between about 120 mmHg and minus 50 mmHg.
